(19) United States
(12) Patent Application Publication
(10) Pub. No.: US 2017/0056356 A1
(43) Pub. Date: Mar. 2, 2017

(54) UTILIZATION OF DIALKYL FUMARATES IN TREATMENT OF HEART FAILURE

(71) Applicant: Vijaykumar Rajasekhar, Apple Valley, CA (US)

(72) Inventor: Vijaykumar Rajasekhar, Apple Valley, CA (US)

(21) Appl. No.: 15/249,744

(22) Filed: Aug. 29, 2016

Related U.S. Application Data
(60) Provisional application No. 62/210,738, filed on Aug. 27, 2015.

(51) Int. Cl. A61K 31/22; A61K 9/20; A61K 9/28; A61K 45/06

(52) U.S. Cl. CPC A61K 31/22; A61K 45/06; A61K 9/20; A61K 9/2833

(57) ABSTRACT

Pharmaceutical preparations and methods for treating or preventing a heart failure disease, including heart failure with preserved ejection fraction (HFPEF), with one or more dialkyl fumarates alone or in combination with one or more second agents are provided herein. In some embodiments, the pharmaceutical preparations are in the form of micro-tablets or pellets.

### CROSS-REFERENCE TO RELATED APPLICATIONS

This patent application claims priority to U.S. provisional patent application Ser. No. 62/210,738, filed on Aug. 27, 2015, which is herein incorporated by reference in its entirety.

### INCORPORATION BY REFERENCE

All publications and patent applications mentioned in this specification are herein incorporated by reference in their entireties, as if each individual publication or patent application was specifically and individually indicated to be incorporated by reference in its entirety.

### TECHNICAL FIELD

The present disclosure relates to the fields of cardiology and pharmaceutical sciences. In particular, the present disclosure relates to certain pharmaceutical preparations comprising dialkyl fumarates and administering said pharmaceutical preparations alone or in combination with one or more second agents for the treatment of a heart failure disease including, heart failure with preserved ejection fraction.

### BACKGROUND

Heart failure (HF) is a major health problem in the United States (U.S.) and elsewhere. In the U.S., HF affects over 5 million people with approximately half a million new cases occurring each year. HF is the leading cause of hospitalizations in people over 65 years in age. HF has many potential causes and diverse clinical features. Symptoms of heart failure can include dyspnea during activity or at rest, cough with white sputum, rapid weight gain, swelling in ankles, legs and abdomen, dizziness, fatigue and weakness, rapid or irregular heartbeats, nausea, palpitations, and chest pains.

About half of heart failure patients have HF with preserved ejection fraction (HFPEF). In traditional HF (i.e., heart failure with reduced ejection fraction (HFREF)), the ventricle cannot contract normally. However in patients with HFPEF, the declined performance of the heart ventricle is not at the time of contraction (systole), but during the relaxation/filling phase of diastole. HFPEF patients show normal ejection fraction of blood pumped out of the ventricle, but the heart muscle does not quickly relax to allow efficient filling of blood returning from the body. Morbidity and mortality of HFPEF are similar to HFREF; however, therapies that treat HFREF are not effective in treating or preventing HFPEF. Patients with HFPEF have an ejection fraction of ≧40%, ≧45%, or ≧50% depending on which definition is chosen from the literature. On the other hand, patients with HFREF have an ejection fraction of either ≦35% or ≦40% depending on which definition and guidelines are used. For ease of simplicity, and not to be limiting in any way, HFPEF can be considered as having an ejection fraction ≧40% and HFREF can be considered as having an ejection fraction <40%.

Other names for the two primary clinical subsets of HF are diastolic heart failure (DHF) and systolic heart failure (SHF). SHF, which is also known as heart failure with reduced ejection fraction (HFREF) involves an abnormality of the heart resulting in failure of the heart to pump blood at a rate needed for metabolizing tissues at rest and/or during exertion. DHF, which is also known as heart failure with preserved ejection fraction (HFPEF), is a clinical syndrome with symptoms and signs of HF, a preserved ejection fraction, and abnormal diastolic function. The clinical manifestations of HFREF and HFPEF have distinct differences in risk factors, patient characteristics, and pathophysiology. Moreover, medications proven effective in HFREF have not been found to be effective in HFPEF. At present there are no approved treatments to reduce mortality in HFPEF.

In HFREF, medications such as beta-blockers, ace-inhibitors, angiotensin receptor blockers, isosorbide dinitrate, hydralazine, aldosterone inhibitors, and angiotensin receptor neprilysin inhibitors have been shown to provide benefit. However, these medications have not shown to be beneficial in patients with HFPEF and are not approved therapies for HFPEF.

Given that there are currently no approved treatments to improve survival in HFPEF, there remains, therefore, a real urgent need for a product that can improve morbidity and mortality of patients with HFPEF.

The present disclosure addresses these needs in patients with HFPEF as well as in patients at risk of developing HFPEF, due to conditions including but not limited to hypertension, diabetes, COPD, atrial fibrillation, obesity, or ischemic heart disease.

### SUMMARY

The present disclosure is directed to methods useful in the treatment of heart failure diseases including, heart failure with preserved ejection fraction (HFPEF), the method comprising administering to the subject a therapeutically effective amount of a compound that upregulates the Nrf2 pathway, inhibits NF-κB, or increases adiponectin. Compositions described herein comprise one or more dialkyl fumarates that are configured for the treatment of a heart failure disease including, heart failure with preserved ejection fraction. In one embodiment, dialkyl fumarate is dimethyl fumarate. In some embodiments, the disclosed pharmaceutical preparations are in the form of micro-tablets or micro-pellets containing dialkyl fumarates.

Additionally, methods disclosed herein provide for administering pharmaceutical preparations containing dialkyl fumarates in combination with one or more second agents that do not upregulate the Nrf2 pathway, inhibit NF-κB, or increase adiponectin for use in the treatment of a heart failure disease. In some embodiments, the second agent may include one or more of: a diuretic, an ace-inhibitor, a beta-blocker, an angiotensin receptor blocker (ARB), isosorbide dinitrate, hydralazine, an angiotensin receptor-neprilysin inhibitor (ARNI), an aldosterone antagonist, a PDE5 inhibitor, a statin, a neprilysin inhibitor, an aldosterone inhibitor, an antitumor necrosis factor-alpha therapy and combination thereof. In one embodiment, the second agent is a statin.

The present disclosure provides for the treatment of a heart failure disease by the administration of one or more dialkyl fumarates of the formula:

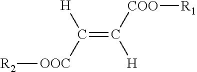

wherein R1 and R2, which may be the same or different, independently represent a linear, branched, or cyclic, saturated or unsaturated C1-20 alkyl radical which may be optionally substituted with halogen (Cl, F, I, Br), hydroxy, alkoxy, nitro, or cyano.

The C1-20 alkyl radicals, preferably C1-8 alkyl radicals, most preferably C1-5 alkyl radicals are, for example, methyl, ethyl, n-propyl, isopropyl, n-butyl, sec-butyl, t-butyl, pentyl, cyclopentyl, 2-ethyl hexyl, hexyl, cyclohexyl, heptyl, cycloheptyl, octyl, vinyl, allyl, 2-hydroxyethyl, 2 or 3-hydroxy propyl, 2-methoxy ethyl, methoxy methyl or 2- or 3-methoxy propyl. In one embodiment, at least one of the radicals or R2 is C1-5 alkyl, especially methyl or ethyl. In one embodiment, R1 and R2 are the same or different C1-5 alkyl radicals such as methyl, ethyl, n-propyl, or t-butyl. In one embodiment, R1 and R2 are identical and are methyl or ethyl. In one embodiment, one or more dialkyl fumarates are dimethyl fumarate, methylethyl fumarate, and diethyl fumarate.

The present disclosure provides method for chronic treatment of heart failure with preserved ejection fraction (HFPEF) by administering to the subject, over the long term or over a prolonged period of time, a daily dose of dimethyl fumarate.

In one embodiment, dimethyl fumarate is administered to the subject in a therapeutically effective amount of about 120 to 720 mg per day and in separate or the same administrations over the long term or over a prolonged period of time.

The present disclosure provides a method for treating heart failure with preserved ejection fraction by a combination therapy, the method comprising: (a) providing simultaneously, separately, or sequentially to a subject a therapeutically effective amount of a first compound that upregulates the Nrf2 pathway, inhibits NF-κB, or increases adiponectin and (b) administering a second agent that does not upregulate the Nrf2 pathway, inhibit NF-κB, or increase adiponectin. In some embodiments, the first compound is dimethyl fumarate and the second agent selected from the group consisting of: a diuretic, an ace-inhibitor, a beta-blocker, an angiotensin receptor blocker, isosorbide dinitrate, hydralazine, an aldosterone inhibitor, an angiotensin receptor neprilysin inhibitor, a PDE5 inhibitor, a statin, a neprilysin inhibitor, an antitumor necrosis factor-alpha therapy as active ingredients, and a combination thereof. In one embodiment, dimethyl fumarate is administered at a dose of 120 mg to 720 mg and a statin is given at a dose of 10 mg to 80 mg.

The present disclosure provides for pharmaceutical preparations used for treatment of heart failure with preserved ejection fraction. The pharmaceutical preparation may include an active ingredient and optionally carriers and excipients. In one embodiment, the active ingredient includes dimethyl fumarate and a second agent selected from group consisting of a beta-blocker, an ace-inhibitor, an angiotensin receptor blocker, isosorbide dinitrate, hydralazine, an aldosterone inhibitor, an angiotensin receptor neprilysin inhibitor, a PDE5 inhibitor, a statin, a neprilysin inhibitor, an antitumor necrosis factor-alpha therapy and combinations thereof; and pharmaceutical preparation is in the form of microtablets and the mean diameter of the microtablets is about 2,000 μm, excluding any coating on the microtablets. In another embodiment, the pharmaceutical preparation includes dimethyl fumarate at a dose of 10 mg to 300 mg and statin at a dose of 10 mg to 80 mg.

### DETAILED DESCRIPTION

The present disclosure relates to the fields of cardiology and pharmaceutical sciences. In particular, the present disclosure relates to certain pharmaceutical preparations comprising dialkyl fumarates and administering said pharmaceutical preparations alone or in combination with one or more second agents for the treatment of a heart failure disease including, heart failure with preserved ejection fraction.

Compositions described herein comprise one or more dialkyl fumarates configured for the treatment of a heart failure disease. In some embodiments the compositions are used for the prevention or treatment of heart failure with preserved ejection fraction (HFPEF). In some embodiments, the disclosed pharmaceutical preparations are in the form of micro-tablets or micro-pellets containing dialkyl fumarates.

The present disclosure provides, in part, methods for the prevention or treatment of a heart failure disease by administering to a subject in need thereof, a therapeutically effective amount of one or more pharmaceutical preparations in the form of micro-tablets or micro-pellets containing dialkyl fumarates.

The heart failure disease may be heart failure with preserved ejection fraction (HFPEF); heart failure with ejection fraction ≧40%; diastolic heart failure; heart failure with elevated levels of TNF-α, IL-6, CRP, or TGF-β; hypertension with a risk of developing HFPEF; atrial fibrillation with a risk of developing HFPEF; diabetes with a risk of developing HFPEF; COPD with a risk of developing HFPEF; ischemic heart disease with a risk of developing HFPEF; obesity with a risk of developing HFPEF; chronic heart failure; compensated heart failure; decompensated heart failure; or other conditions known to have a high risk of developing HFPEF

Patients with heart failure can be divided into those with (1) heart failure with a reduced ejection fraction (HFREF) and (2) heart failure with a preserved ejection fraction (HFPEF). All of these patients, regardless of ejection fraction status (EF value), have the clinical syndrome of heart failure. In addition, many features are similar across the EF spectrum, including frequent hospitalization and reduced survival. Patients with HFPEF have a devastating 5-year mortality rate (approaching 60%), costly morbidity (6-month hospitalization rate of 50%), and debilitating symptoms (maximum myocardial oxygen consumption [MVo₂] averaging 14 mL/g/min). Clear differences are also recognized between HFPEF and HFREF. Compared with those with HFREF, patients with HFPEF are typically older and more likely to be female; however, HFPEF does occur in both men and women throughout the fifth to the ninth decades of life. The most common disease leading to HFPEF is systolic hypertension, which is present in more than 85% of patients. Differences in cardiovascular structure and function between HFPEF and HFREF also are well recognized. Patients with HFPEF have normal LV end-diastolic volume and normal (or near-normal) EF and stroke volume and commonly exhibit concentric remodeling of either the left ventricular (LV) chamber and/or cardiomyocytes. Finally, differences are also evident in the effects of pharmacologic treatment in patients with HFREF versus HFPEF. Standard heart failure therapy shown to be effective in HFREF has not been found to reduce morbidity or mortality associated with HFPEF, leaving a substantial area of unmet need, which the present disclosure addresses with the use of dialkyl fumarates, such as dimethyl fumarate (DMF), to treat or prevent heart failure diseases.

Drugs that have previously shown mortality benefit for HFREF but not for HFPEF include beta-blockers, ACE-inhibitors, angiotensin receptor blockers, isosorbide dinitrate, hydralazine, angiotensin receptor-neprilysin inhibitors, and aldosterone antagonists. While there are many drugs available to treat patients with HFREF, the only strategy for HFPEF remains managing volume status with diuretics and managing co-morbidity conditions. Due to lack of therapeutic options, HFPEF remains deadly.

The pathophysiology of HFPEF is still not fully understood. A recent hypothesis of HFPEF, as described by Zuo et al, traces its roots to a proinflammatory state initiated in part by the existence of comorbidities (e.g., hypertension, diabetes mellitus, vasculopathy, renal disease, metabolic syndrome, and atrial fibrillation) that create a favorable environment for the production of reactive oxygen species (ROS). Triggering a cascade that involves reduced nitric oxide (NO) availability and elevated ROS levels in the coronary endothelium that eventually contribute to hypertrophy and increased resting tension in cardiomyocytes (J Appl Physiol. 2015, 119(8), 944-951). In HFPEF patients, ROS produced during comorbidity-induced endothelial inflammation may trigger a signaling cascade involving NO that ultimately increases interstitial fibrosis and activates cardiac remodeling. These actions contribute to the hallmarks of HFPEF: ventricular stiffness, impaired relaxation and cardiac dysfunction.

Based on Zuo et al hypothesis of inflammation as a culprit in HFPEF, a number of treatments to reduce inflammation have been explored. Anti-inflammatory treatments including antitumor necrosis factor-alpha therapies and 3-hydroxy-3-methylglutaryl coenzyme A reductase inhibitors (statins) have in general failed to improve clinical outcomes. Although a small retrospective, nonrandomized study suggested potential benefits to statin treatment in HFPEF, no benefit was seen in the subgroup of patients with preserved EF enrolled in the large, prospective GISSI-HF (i.e., Gruppo Italiano per lo Studio della Sopravvivenza nell'I nfarto Miocardico-Heart Failure) trial, which randomized patients to rosuvastatin or placebo. Angiotensin receptor blockers (ARB) also reduce markers of inflammation and improve endothelial function, but they have been ineffective in improving outcomes in HFPEF patients in randomized trials.

Paulus and Tschope (J Am Coil Cardiol, 2013, 62, 263-271) postulate that comorbidities in HFPEF (e.g., obesity, hypertension, diabetes, chronic obstructive pulmonary disease, anemia, and chronic kidney disease) conspire to create a systemic inflammatory state that promotes coronary microvascular endothelial dysfunction. Endothelial inflammation results in generation of reactive oxygen species and reduces NO bioavailability, which in turn results in lower levels of cyclic guanosine monophosphate (cGMP) and lower activity of protein kinase G (PKG). Declining PKG activity accelerates pro-hypertrophic signaling and increases myocyte stiffness by promoting hypophosphorylation of titin, enhancing diastolic dysfunction and ventricular stiffening characteristics of HFPEF.

Based on this hypothesis, it was hypothesized that sildenafil, which raises myocardial PKG activity by inhibiting breakdown of cGMP by phosphodiesterase 5, may improve pulmonary artery pressures, right ventricular function, and LV relaxation and distensibility in HFPEF. However, sildenafil did not impact the primary outcome of functional capacity in HFPEF patients in the RELAX trial (i.e., Phosphodiesterase-5 Inhibition to Improve Clinical Status and Exercise Capacity in Diastolic Heart Failure). Thus, there still remains an urgent need for new therapies to treat HFPEF.

Another possible candidate for treating HFPEF is LCZ696, a combined angiotensin receptor neprilysin inhibitor (ARNI) that has been recently shown to reduce mortality in HFREF but not yet in HFPEF patients. LCZ696 inhibits natriuretic peptide breakdown and enhances cGMP activation, and in HFPEF was associated with incremental reductions in circulating N-terminal pro-B-type natriuretic peptide (NT-proBNP) levels when compared to treatment with the ARB valsartan alone. However, these reductions were incremental, and it is yet to be seen whether LCZ696 or other angiotensin receptor-neprilysin inhibitors will lead to any significant mortality or clinical benefit in HFPEF patients.

Another HFREF drug spironolactone, an aldosterone antagonist, was shown to be ineffective for HFPEF in the TOPCAT trial (i.e., Treatment of Preserved Cardiac Function Heart Failure with an Aldosterone Antagonist). In 3,445 patients with symptomatic heart failure and a preserved ejection fraction, treatment with spironolactone did not significantly reduce the incidence of the primary composite outcome of death from cardiovascular causes, cardiac arrest, or hospitalization for the management of heart failure.

Thus, despite many attempts to treat the deadly HFPEF condition, a beneficial treatment to improve HFPEF remains elusive. The present disclosure fills this unmet need by providing a novel treatment for patients with a heart failure disease, including HFPEF.

The present disclosure is directed to the surprising and unexpected discovery that using dialkyl fumarates, alone or in combination with one or more second agents, is beneficial for the treatment of a heart failure disease. The object of the present disclosure is achieved by the use of certain pharmaceutical preparations comprising dialkyl fumarates and administering said pharmaceutical preparations alone or in combination with one or more second agents for the treatment of a heart failure disease. In some embodiments, the compositions are used for the prevention or treatment of heart failure with preserved ejection fraction (HFPEF). In some embodiments, the disclosed pharmaceutical preparations are in the form of micro-tablets or micro-pellets containing dialkyl fumarates.

In some embodiments, at least one dialkyl fumarate is combined with one or more compounds that reduce ROS, increase the availability of NO, increase cGMP, or increase PGK.

In some embodiments, at least one dialkyl fumarate is combined with one or more second agents selected from group consisting of a diuretic, an ace-inhibitor, a beta-blocker, an angiotensin receptor blocker (ARB), isosorbide dinitrate, hydralazine, an angiotensin receptor-neprilysin inhibitor (ARNI), an aldosterone antagonist, a PDE5 inhibitor, a statin, a neprilysin inhibitor, an aldosterone inhibitor, an antitumor necrosis factor-alpha therapy, and combination thereof.

In some embodiments, pharmaceutical preparations in the form of micro-tablets and micro-pellets comprising dialkyl fumarates are administered in combination with one or more compounds that reduce ROS, increase the availability of NO, increase cGMP, or increase PGK.

In some embodiments, pharmaceutical preparations in the form of micro-tablets and micro-pellets comprising dialkyl fumarates administered in combination with a diuretic, an ace-inhibitor, a beta-blocker, an angiotensin receptor blocker (ARB), isosorbide dinitrate, hydralazine, an angiotensin receptor-neprilysin inhibitor (ARNI), an aldosterone antagonist, a PDE5 inhibitor, a statin, a neprilysin inhibitor, an aldosterone inhibitor, an antitumor necrosis factor-alpha therapy, or a combination thereof, in order to treat or prevent a heart failure disease, including HFPEF.

Fumaric acid esters (FAEs), such as dialkyl fumarates like DMF, are approved for the treatment of psoriasis and multiple sclerosis, and have been proposed for use in treating a number of immunological, autoimmune, and inflammatory diseases. However, until the present disclosure, fumaric acid esters (FAEs), such as dialkyl fumurates like DMF, have not been explored as a therapy for HFPEF and other heart failure diseases.

FUMADERM®, an enteric coated tablet containing a salt mixture of monoethyl fumarate and dimethyl fumarate (DMF) which is rapidly hydrolyzed to monomethyl fumarate, regarded as the main bioactive metabolite, was approved in Germany in 1994 for the treatment of psoriasis. FUMADERM® is dosed three times a day (TID) with 1-2 grams/day administered for the treatment of psoriasis. FUMADERM® exhibits a high degree of interpatient variability with respect to drug absorption, and food strongly reduces bioavailability. Absorption is thought to occur in the small intestine with peak levels achieved 5-6 hours after oral administration. Significant side effects occur in 70-90% of patients (Brewer and Rogers, Clin Expt'l Dermatology 2007, 32, 246-49; and Hoefnagel et al., Br J Dermatology 2003, 149, 363-369). Side effects of current FAE therapy include gastrointestinal upset including nausea, vomiting, diarrhea, and/or transient flushing of the skin.

Dimethyl fumarate (DMF) is the active component of BG-12, also known as Tecfidera®, studied for the treatment of relapsing-remitting MS (RRMS). In a Phase III RRMS study, BG-12 significantly reduced the proportion of patients who had a relapse, the annualized relapse rate, the rate of disability progression, and the number of lesions on MRI.

The exact mechanisms by which DMF exerts its clinical efficacy in multiple sclerosis is unknown, but some of these effects are believed to be mediated through activation of the nuclear factor (erythroid-derived 2)-like 2 (NRF2) pathway, an endogenous defense mechanism against toxic cell stress. Under basal conditions, NRF2 is sequestered in the cytoplasm by the actin-associated protein kelch-like ECH-associated protein 1 (KEAP1), which targets NRF2 for ubiquitination and subsequent proteasomal degradation. However, in the presence of electrophiles or oxidative stress, these molecules can bind KEAP1 cysteine residues resulting in an allosteric conformational change that diminishes KEAP1-dependent degradation of NRF2. This allows NRF2 to accumulate and translocate to the nucleus, where NRF2 binds to the antioxidant responsive element (ARE), a cis-acting regulatory element that increases expression of detoxifying enzymes and antioxidant proteins. Various synthetic and naturally occurring compounds possessing electrophilic properties, including DMF, have been shown to modify specific cysteine residues on KEAP1 and subsequently activate NRF2.

One explanation for the beneficial effects of BG-12 in multiple sclerosis is that one pathway of FAEs, such as dialkyl fumarates like DMF, is the upregulation of NRF2, which increases expression of ARE, which increases expression of detoxifying enzymes and antioxidant proteins.

Along with research supporting NRF2 activation as a cytoprotective mechanism of DMF, other studies have hypothesized indirect regulation of NRF2 or NRF2-independent mechanisms of action for DMF, including activation of the hydroxycarboxylic acid receptor 2 (HCA2), inhibition of nuclear factor kappa-light-chain-enhancer of activated B cells (NF-κB), activation of hypoxia-inducible factor 1-alpha (HIF1A) and modulation of cellular glutathione (GSH). As α, β carboxylic acid unsaturated esters, fumarates are capable of interacting with various free cysteine residues by Michael addition including those present on the antioxidant thiol, GSH. Schmidt and colleagues have shown that DMF can stably bind GSH and rapidly deplete circulating levels. Other groups have suggested that this intracellular depletion contributes to the anti-inflammatory, immunosuppressive, and cytoprotective properties of DMF. It is therefore likely that multiple mechanisms underlie the immunomodulatory and neuroprotective effects of DMF, including but not limited to NRF2/ARE activation.

As described by Mudd and Kass, there is a preference for oxidation of fatty acids over glucose in a healthy heart (Nature, 2008; 451, 919-928). When the heart is pressure or volume overloaded, there may be a shift towards increased glucose oxidation, which is coupled to a decline in production of the transcriptional coactivator PGC1α. PGC1α modulates the expression of the transcription factors PPAR-α, ERR-α, NRF1, and NRF2, which affect mitochondrial biogenesis and fatty-acid oxidation.

As described by Li et al, NRF2 deficiency, demonstrated by NRF2 knockout in murine models, results in an earlier onset of cardiac dysfunction induced by pressure and volume overload (Arterioscler Thromb Vase Biol. 2009, 29(11), 1843-50).

Zhou et al describes the role of the NRF2-mediated pathway in cardiac remodeling and heart failure (HF). HF and many of the conditions that predispose one to HF are associated with oxidative stress. Increased generation of reactive oxygen species (ROS) in the heart can directly lead to increased necrosis and apoptosis of cardiomyocytes, which subsequently induce cardiac remodeling and dysfunction. NRF2 is a transcription factor that controls the basal and inducible expression of a battery of antioxidant genes and other cytoprotective phase II detoxifying enzymes that are ubiquitously expressed in the cardiovascular system. NRF2 and its target genes may be critical regulators of cardiovascular homeostasis via the suppression of oxidative stress, which is the key player in the development and progression of HF. Zhou et al proposes certain NRF2 activators as therapeutic targets to reduce cardiac remodeling such as sulforaphane, curcumin, carbobenzoxy-Leu-Leu (MG132), resveratrol, garlic organosulfur compounds, allicin, 4-hydroxynonenal (4-HNE), α-lipoic acid, hydrogen sulfate, and 17α-estradiol, but does not include FAEs, dialkyl fumurates, or DMF as revealed by the present disclosure.

The present disclosure discloses the use of fumaric acid esterases (FAEs), such as dialkyl fumarates like DMF, for the treatment of a heart failure disease, including HFPEF.

In acute ischemia due to myocardial infarction, Ashrafian et. al proposes that fumarates are cardioprotective in acute situations via activation of the NRF2 pathway (Cell Metab. 2012, 15(3), 361-71). However, Ashrafian et al claim that fumarates are harmful in chronic situations, including heart failure. The present disclosure counters Ashrafian et al. The present disclosure has determined that FAEs, such as dialkyl fumarates like DMF, may be surprisingly beneficial in the treatment of heart failure diseases, including HFPEF over the long term or during a prolonged period of time with a daily dose.

In some embodiments, long term administration means delivery of the drug in a regular interval during a pre-determined length or for as long as the subject's condition requires the drug. In some embodiments, the long term administration means administration with a drug holiday, for example, a daily dose given for one month, then two weeks off, and then a daily dose give for one month, etc.

Bardoxolone Methyl, also known as RTA 402, CDDO-methyl ester, and CDDO-Me, also is an activator of the NRF2 pathway and inhibits NF-κB. Bardoxolone Methyl was tested in humans as a treatment for chronic kidney disease (CKD) but was found to increase rates of heart failure, and the trial was stopped. Thus, it is surprising that DMF, a NRF2 activator, may be an effective therapy for treatment of HFPEF.

Further, Fumaderm was tested in a short term study (16 weeks) in psoriasis patients and was surprisingly found to increase adiponectin, a protein involved in regulating glucose levels as well as fatty acid breakdown (Schmieder et al., “Impact of fumaric acid esters on cardiovascular risk factors and depression in psoriasis: a prospective pilot study. Arch Dermatol Res, 2015, 307: (5), 413-424). Adiponectin may have cardioprotective properties, but such benefits are still hypothetical. In animal models, adiponectin deficiency has shown to worsen HFPEF, but it is still unknown whether increasing adiponectin will improve outcomes in HFPEF in humans (Tanaka et al., “Effects of Adiponectin on Calcium-Handling Proteins in Heart Failure With Preserved Ejection Fraction.” 2014 Circ Heart Fail, 7, 976-985.). Thus DMF, which increases adiponectin, may provide a therapeutic benefit in HFPEF.

Furthermore, pro-inflammatory cytokines IL-6 and TNF-α are raised in HFPEF, which may lead to increased activity of VCAM, E-Selection, and NADPH oxidase, which increase ROS in coronary microvasculature endothelial cells, leading to the hallmarks of HFPEF: ventricular stiffness, impaired relaxation, and cardiac dysfunction. The present disclosure includes that DMF may reduce damage of ROS in heart failure by multiple pathways including increasing the NRF2/ARE pathway, and possibly by reducing NFkB, which reduces IL-6 and TNF-α.

The present disclosure includes the use of FAEs, such as dialkyl fumarates like DMF, for the treatment of a heart failure disease. The full mechanism of fumaric acids is not completely understood, however, one pathway includes increased activity of the NRF2/ARE pathway. The present disclosure is directed to the surprising and unexpected discovery that using dialkyl fumarates (e.g., those that upregulate the Nrf2 pathway, inhibit NFκB, or increase adiponectin), alone or in combination with other compounds (e.g., those that do not upregulate the Nrf2 pathway, inhibit NFκB, or increase adiponectin), is beneficial for the therapy of a heart failure disease.

The object of the present disclosure is achieved by the use of certain pharmaceutical preparations comprising dialkyl fumarates and administering said pharmaceutical preparations for the treatment of a heart failure disease. In some embodiments the compositions are used for the prevention or treatment of heart failure with preserved ejection fraction (HFPEF). In some embodiments, the disclosed pharmaceutical preparations are in the form of micro-tablets or micro-pellets containing dialkyl fumarates.

The preparations according to the present disclosure do not contain any free fumaric acids per se.

Fumaric acid, for example, inhibits the growth of the Ehrlich ascites tumor in mice, reduces the toxic effects of mitomycin C and aflatoxin, and displays anti-psoriatic and anti-microbial activity. When administered parenterally, transdermally, and especially perorally, high dosages of fumaric acid or its derivatives known so far such as dihydroxyl fumaric acid, fumaramide, and fumaronitrile have such unacceptably severe side effects and high toxicity that, in most cases, such a therapy had to be abandoned in the past.

European Patent Application 0188 749, the disclosure of which is incorporated by reference in its entirety, already describes fumaric acid derivatives and pharmaceutical compositions containing the same for the treatment of psoriasis. Pharmaceutical compositions for the treatment of psoriasis containing a mixture of fumaric acid and other fumaric acid derivatives are known from DE-A-25 30 372, the disclosure of which is incorporated by reference in its entirety.

DE-A-26 21 214, the disclosure of which is incorporated by reference in its entirety, describes medicaments containing the fumaric acid monoethyl ester and its mineral salts as active ingredients for the treatment of psoriasis. The publication “Hautarzt (Dermatologist) (1987) 279-285” discusses the use of fumaric acid monoethyl ester salts. Pharmaceutical preparations containing a mixture of fumaric acid monoalkyl ester salts and a fumaric acid diester for the treatment of psoriasis, psoriatic arthritis, neurodermatitis, and enteritis regionalis Crohn are known from EP 0 312 697 B1 the disclosure of which is incorporated by reference in its entirety.

Specifically, the object of the present disclosure is achieved by the use of one or more dialkyl fumarates of the formula:

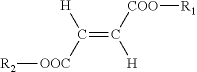

wherein R1 and R2, which may be the same or different, independently represent a linear, branched, or cyclic, saturated or unsaturated C1-20 alkyl radical which may be optionally substituted with halogen (Cl, F, I, Br), hydroxy, alkoxy, nitro, or cyano for preparing a pharmaceutical preparation for use in a composition for the treatment of a heart failure disease.

The C1-20 alkyl radicals, preferably C1-8 alkyl radicals, most preferably C1-5 alkyl radicals are, for example, methyl, ethyl, n-propyl, isopropyl, n-butyl, sec-butyl, t-butyl, pentyl, cyclopentyl, 2-ethyl hexyl, hexyl, cyclohexyl, heptyl, cycloheptyl, octyl, vinyl, allyl, 2-hydroxyethyl, 2 or 3-hydroxy propyl, 2-methoxy ethyl, methoxy methyl, or 2- or 3-methoxy propyl. In one embodiment, at least one of the radicals or R2 is C1-5 alkyl, especially methyl or ethyl. In one embodiment, R1 and R2 are the same or different C1-5 alkyl radicals such as methyl, ethyl, n-propyl, or t-butyl. In one embodiment, R1 and R2 are identical and are methyl or ethyl. In one embodiment, one or more dialkyl fumarates are dimethyl fumarate, methyl ethyl fumarate, and diethyl fumarate.

The dialkyl fumarates to be used according to the present disclosure are prepared by processes known in the art (see, for example, EP 0 312 697, the disclosure of which is incorporated by reference in its entirety).

For example, the active ingredients are used for preparing oral preparations in the form of tablets, micro-tablets, pellets, or granulates, optionally in capsules or sachets. Preparations in the form of micro-tablets or pellets, optionally filled in capsules or sachets are also a subject matter of the present disclosure. The oral preparations may be provided with an enteric coating. Capsules may be soft or hard gelatin capsules.

The dialkyl fumarates used according to the present disclosure may be used alone or as a mixture of several compounds, optionally in combination with the customary carriers and excipients. In some embodiments, the amounts to be used are selected in such a manner that the preparations obtained contain the active ingredient in an amount corresponding to 10 to 300 mg of fumaric acid.

In some embodiments, the preparations according to the present disclosure comprise a total amount of 10 to 300 mg of dimethyl fumarate and/or diethyl fumarate.

In some embodiments, the size or the mean diameter, respectively, of the pellets or micro-tablets is in the range from 300 to 2,000 μm, and in one embodiment, in the range of 500 or 1,000 μm.

In addition to the preparations for peroral administration in the form of micro-pellets, micro-tablets, capsules (such as soft and hard gelatine capsules), granulates, and tablets cited above, suitable pharmaceutical preparations are preparations for cutaneous and transdermal administration in the form of ointments, plasters, lotions, or shower preparations and for parenteral administration in the form of aqueous micro-dispersions, oil-in-water emulsions, or oily solutions for rectal administration of suppositories or micro-enemas. Pharmaceutical preparations in the form of micro-tablets or micro-pellets are preferred for the therapy of a heart failure disease.

According to the present disclosure, administration of dialkyl fumarates may also be carried out in combination with administration of one or more preparations of other heart failure medications, such as but not limited to diuretics, ace-inhibitors, beta-blockers, angiotensin receptor blockers, vasodilators, angiotensin receptor-neprilysin inhibitors, aldosterone antagonists, or combinations thereof. For this purpose, the preparations administered may contain a combination of the active ingredients in the known dosages or amounts, respectively. Likewise, the combination therapy may comprise the parallel administration of separate preparations, by the same or different routes. Optionally, the dosage of the active ingredient contained in addition to the dose of the fumaric acid derivative administered in accordance with the present disclosure may be reduced advantageously.

Dialkyl fumarates, acting on the NRF2/ARE pathway, work on a separate portion of the anti-oxidant cascade compared to other anti-oxidant compounds such as PDE5 inhibitors, statins, ARNIs, aldosterone inhibitors, and antitumor necrosis factor-alpha therapies. Thus, when dialkyl fumarates are used in combination with at least one of these compounds, there happens to be an unexpected synergistic effect beneficial in the treatment of a heart failure disease. According to the present disclosure, administration of dialkyl fumarates may also be carried out in combination with the administration of one or more pharmaceutical preparations with known anti-oxidant properties, such as but not limited to PDE5 inhibitors, statins, angiotensin receptor-neprilysin inhibitors (ARNI), angiotensin receptor blockers (ARB), neprilysin inhibitors, aldosterone inhibitors, antitumor necrosis factor-alpha therapy or combination thereof. For this purpose, the preparations administered may contain a combination of the active ingredients in the known dosages or amounts, respectively. Likewise, the combination therapy may comprise the parallel administration of separate preparations, by the same or different routes. Optionally, the dosage of the active ingredient contained in addition to the dose of the fumaric acid derivative administered in accordance with the present disclosure may be reduced advantageously.

In one embodiment, dimethyl fumarate (DMF) is administered either separately or together with a statin. In another embodiment, a 120 mg or 240 mg dose of DMF is given daily, twice daily (BID), or three times daily (TID) to a patient with a statin dosage between 10 mg to 80 mg. In another embodiment, dimethyl fumarate is administered at a dose of 120 mg to 720 mg and a statin is given at a dose of 10 mg to 80 mg.

In yet another embodiment the statin is selected from group consisting of atorvastatin, fluvastatin, lovastatin, pravastatin, rosuvastatin, and simvastatin.

In one embodiment, dimethyl fumarate (DMF) is administered either separately or together with an angiotensin receptor-neprilysin inhibitor (ARNI) and optionally a statin. In one embodiment, the angiotensin receptor-neprilysin inhibitor (ARNI) is LCZ696 (combination of valsartan and sacubitril) and the statin is selected from group consisting of atorvastatin, fluvastatin, lovastatin, pravastatin, rosuvastatin, and simvastatin. In yet another embodiment, a 120 mg or 240 mg dose of DMF is given daily, BID, or TID, to a patient with LCZ696 50 mg to 400 mg daily and optionally a statin dosage between 10 mg to 80 mg.

By administration of the dialkyl fumarates in the form of micro-tablets, in one embodiment, gastrointestinal irritations and side effects, which are reduced already when conventional tablets are administered but is still observed, may be further reduced when using fumaric acid derivatives and salts.

It is presumed that, upon administration of conventional tablets, the ingredients of the tablet are released in the intestine in a concentration which is too high, causing local irritation of the intestinal mucous membrane. This local irritation results in a short-term release of very high TNF-α concentrations, which may be responsible for the gastrointestinal side effects. In the case of enteric-coated micro-tablets in capsules, very low local concentrations of the active ingredients in the intestinal epithelial cells are achieved. The micro-tablets are incrementally released by the stomach and passed into the small intestine by peristaltic movements so that distribution of the active ingredients is improved.

This means that enteric-coated micro-tablets in the same dosage are distributed already in the stomach and passed to the intestine in portions where the active ingredients are released in smaller dosages. This avoids local irritation of the intestinal epithelial cells and the release of TNF-α. It is assumed that this results in the improved tolerance of micro-tablets in the gastrointestinal tract versus conventional tablets.

In addition, resorption is improved, because the dialkyl fumarates to be used according to the present disclosure are not the active ingredient per se, but a so-called prodrug, which must be converted into the active ingredient in the body.

### Pharmaceutical Preparation Example

A pharmaceutical composition is formulated to be compatible with its intended route of administration. Methods to accomplish the administration are known in the art.

In principle, the oral preparations according to the present disclosure are in the form of tablets or micro-tablets prepared by classical tabletting processes. Alternatively, other methods for the preparation of tablets may be used, such as direct tabletting and processes for preparing solid dispersions in according with the melt method and the spray drying method. The tablets may be provided with an enteric coating. The enteric coating may be applied in a classical coating pan or sprayed on or applied in a fluidised-bed apparatus. The tablet may also be provided with a film coat. Examples of some of formulations containing DMF are given in U.S. Pat. No. 6,509,376, the disclosure of which is herein incorporated by reference in its entirety.

### Prophetic Example 1

The following prophetic example serves to provide approximate dosage levels of dimethyl fumarate to achieve the intended effect, for example treatment of chronic heart failure with preserved ejection fraction (HFPEF). Based on the literature, a few assumptions about the dosage can be made, as will be described in further detail below.

The full mechanism of fumaric acid esters such as dimethyl fumarate (DMF) and its primary metabolite, monomethyl fumarate (MMF), is not completely understood, but their beneficial effects appear to be mediated, at least in part, through the activation of the NRF2 antioxidant response pathway, which further increases expression of ARE, which increases expression of detoxifying enzymes and antioxidant proteins.

NRF2 deficiency, demonstrated by NRF2 knockout in murine models, results in an earlier onset of cardiac dysfunction induced by pressure and volume overload (Li et al Arterioscler Thromb Vase Biol. 2009, 29(11), 1843-50). Certain NRF2 activators such as sulforaphane, curcumin, carbobenzoxy-Leu-Leu (MG132), resveratrol, garlic organosulfur compounds, allicin, 4-hydroxynonenal (4-HNE), α-lipoic acid, hydrogen sulfate, and 17α-estradiol have been used as therapeutic targets to reduce cardiac remodeling, but dimethyl fumarate have not been used yet to reduce cardiac remodeling (Zhou et al; J Appl Physiol. 2015, 119(8), 944-951).

Fumarates are cardioprotective in acute situations via activation of the NRF2 pathway in acute ischemia due to myocardial infarction (Ashrafian et. al; Cell Metab. 2012, 15(3), 361-71). However, Ashrafian et. al claims that fumarates are harmful in chronic situations, including heart failure.

Dimethyl Fumarate has been tested for multiple sclerosis and psoriasis at multiple dosages in the past, including 120 mg, 240 mg, daily, BID, and TID. The side effect profile was similar regardless of which dosage was used. In order to determine dosage of DMF for HFPEF, various levels of DMF (120 mg, 240 mg, daily, BID, and TID) will be administered to patients. In one embodiment, dimethyl fumarate is administered at a dose of 120 mg to 720 mg.

Dimethyl fumarate has never been tested chronically in humans to see long term cardiac effects, for example, for chronic treatment of HFPEF. A daily dose of dimethyl fumarate will be administered over the long term or over a prolonged period of time (e.g., one month, two months, three months, four months, five months, six months, seven months, eight months, nine months, ten months, eleven months, twelve months, one year, two years, three years, four years, five years, ten years, fifteen years, twenty years, etc.). In some embodiments, long term administration may include a drug holiday, for example, daily dose given for one month, two weeks off, one month on etc.

Furthermore, pro-inflammatory cytokines IL-6 and TNF-α are raised in HFPEF, which may lead to increase activity of VCAM, E-Selection, and NADPH oxidase, which increase ROS in coronary microvasculature endothelial cells, leading to the hallmarks of HFPEF: ventricular stiffness, impaired relaxation, and cardiac dysfunction. The prodrugs of monomethyl fumarate may reduce damage of ROS in heart failure by multiple pathways including increasing the NRF2/ARE pathway, and possibly by reducing NF-kB, which reduces IL-6 and TNF-α.

LCZ696, a combined angiotensin receptor neprilysin inhibitor (ARNI), has recently shown to reduce mortality in HFREF but not yet in HFPEF patients. LCZ696 inhibits natriuretic peptide breakdown and enhances cGMP activation, and in HFPEF was associated with incremental reductions in circulating N-terminal pro-B-type natriuretic peptide (NT-proBNP) levels when compared to treatment with the ARB valsartan, alone. However, these reductions were incremental, and it is yet to be seen whether LCZ696 or other angiotensin receptor-neprilysin inhibitors will lead to any significant mortality or clinical benefit in HFPEF patients. Furthermore, the comparison with ARB valsartan alone is flawed in that ARB valsartan is used in the treatment of HFREF but not in HFPEF.

The patient's baseline TNF-alpha, IL-6, NT-proBNP will be measured at the start of the trial and compared to levels at various intervals (weeks to months to years) to determine the ideal dosage based on reductions in TNF-alpha, IL-6, and/or NT-proBNP as a quantitative endpoint. Other endpoints will be compared as well, such as NYHA Heart Failure class, hospital visits and admissions, and need for other heart failure medications. Once a proposed dosage is chosen (120 mg vs 240 mg, daily, BID, or TID), such a dosage will then be tested in a larger group of HFPEF patients to measure changes in morbidity and mortality.

### Prophetic Example 2

Based on the above prophetic example, an exemplary, non-limiting embodiment is described in detail below. As described herein, a user may include a male or female between the ages of 50 to 100 with ejection fraction of greater than 40%, and more likely to be a female with a documented history of high blood pressure, diabetes, and/or COPD; with at least one episode of fluid overload; or who has HFPEF or is at risk of developing (HFPEF).

The most common disease leading to HFPEF is systolic hypertension, which is present in more than 85% of patients. Patients with HFPEF have normal LV end-diastolic volume and normal (or near-normal) EF and stroke volume and commonly exhibit concentric remodeling of either the LV chamber and/or cardiomyocytes. Patients with HFPEF have a devastating 5-year mortality rate (approaching 60%), costly morbidity (6-month hospitalization rate of 50%), and debilitating symptoms (maximum myocardial oxygen consumption [MVO2] averaging 14 mL/g/min).

More than half of heart failure patients have heart failure with preserved ejection fraction (HFPEF). Morbidity and mortality of HFPEF are similar to traditional HF; however, medications proven effective in HFREF have not been found to be effective in HFPEF. At present there are no approved treatments to reduce mortality in HFPEF. In HFREF, medications such as beta-blockers, ace-inhibitors, angiotensin receptor blockers, isosorbide dinitrate, hydralazine, aldosterone inhibitors, and angiotensin receptor neprilysin inhibitors have been shown to provide benefit. However, these medications have not shown to be beneficial in patients with HFPEF, and are not approved therapies for HFPEF.

### Prophetic Example 3

The following prophetic example serves to provide a combination therapy for patients with HFPEF, which includes DMF with a statin. To date, there has been no prospective study of statins in patients with HFPEF. However, statins have pleotropic effects, in which they have been shown to be beneficial to non-HFPEF patients beyond what is predicted based on their ability to reduce cholesterol, likely through anti-inflammatory pathways. By combining a statin with a DMF, we expect a synergistic effect to reduce the reactive oxygen species associated with HFPEF, which in turn will reduce stiffness in HFPEF and also reduce biomarkers such as IL-6, TNF-alpha, or NT-proBNP, ultimately improving cardiac remodeling and survival in HFPEF patients. In one such example, a 120 mg or 240 mg dose of DMF is given daily, BID, or TID to a patient with a statin dosage between 10 mg to 80 mg. In another embodiment, dimethyl fumarate is administered at a dose of 120 mg to 720 mg and a statin at a dose of 10 mg to 80 mg.

What is claimed is:

1. A method of treating a heart failure disease, in a subject in need thereof, the method comprising administering to the subject a therapeutically effective amount of a compound that upregulates the Nrf2 pathway, inhibits NFκB, or increases adiponectin.

2. The method of claim 1, wherein the heart failure disease is heart failure with preserved ejection fraction (HFPEF).

3. The method of claim 1, wherein the compound comprises one or more dialkyl fumarates of the formula: 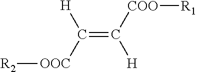
wherein R1 and R2, which may be the same or different, independently represent a linear, branched, or cyclic, saturated or unsaturated C1-20 alkyl radical which may be optionally substituted with halogen (Cl, F, I, Br), hydroxy, C1-4 alkoxy, nitro, or cyano.

4. The method of claim 3, wherein the dialkyl fumarate is dimethyl fumarate.

5. The method of claim 1, wherein a pharmaceutical preparation is administered to the subject, wherein said pharmaceutical preparation comprises 10 to 300 mg of dimethyl fumarate.

6. The method of claim 1, wherein the dimethyl fumarate is administered in the form of tablets or microtablets in capsules.

7. The method of claim 1, wherein dimethyl fumarate is administered to the subject in a therapeutically effective amount of about 120 to 720 mg per day and in separate administrations over the long term.

8. The method of claim 1, wherein the dialkyl fumarate is administered in combination with a second agent that does not upregulate the Nrf2 pathway, inhibit NFκB or increase adiponectin and wherein the second agent is useful for treating heart failure disease.

9. The method of claim 8, wherein the second agent is selected from the group consisting of: a diuretic, an ace-inhibitor, a beta-blocker, an angiotensin receptor blocker (ARB), isosorbide dinitrate, hydralazine, an angiotensin receptor-neprilysin inhibitor (ARNI), an aldosterone antagonist, a PDE5 inhibitor, a statin, a neprilysin inhibitor, an aldosterone inhibitor, an antitumor necrosis factor-alpha therapy, and combination thereof.

10. The method of claim 9, wherein the second agent is the statin.

11. A pharmaceutical preparation in the form of microtablets or micropellets comprising one or more dialkyl fumarates of the formula: 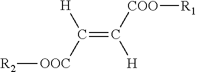
wherein R1 and R2, which may be the same or different, independently represent a linear, branched, or cyclic, saturated or unsaturated C1-20 alkyl radical which may be optionally substituted with halogen (Cl, F, I, Br), hydroxy, C1-4 alkoxy, nitro, or cyano, and optionally suitable carriers and excipients for use in therapy of heart failure with preserved ejection fraction.

12. The preparation of claim 11, wherein the one or more dialkylfumarates is dimethylfumarate.

13. The preparation of claim 11, wherein an amount of the dialkylfumarate in said preparation corresponds to 10 to 300 mg of fumaric acid.

14. The preparation of claim 11, wherein the preparation is formulated into an oral preparation in which the microtablets or micropellets are in capsules or sachets.

15. The preparation of claim 11, wherein the microtablets or micropellets are provided with an enteric coating.

16. The preparation of claim 11, wherein the preparation is formulated into an oral preparation in which the microtablets or micropellets are in capsules or sachets, and wherein the amount of dialkylfumarate in said preparation corresponds to 10 to 300 mg of fumaric acid.

17. The preparation of claim 11, wherein the microtablets or micropellets are provided with an enteric coating and wherein the amount of dialkylfumarate in said preparation corresponds to 10 to 300 mg of fumaric acid.

18. The preparation of claim 11, wherein the preparation further comprise one or more second agents selected from group consisting of: a diuretic, an ace-inhibitor, a beta-blocker, an angiotensin receptor blocker, isosorbide dinitrate, hydralazine, an angiotensin receptor-neprilysin inhibitor, an aldosterone antagonist, a PDE5 inhibitor, a statin, an angiotensin receptor-neprilysin inhibitor, an angiotensin receptor blocker, a neprilysin inhibitor, an aldosterone inhibitor, an antitumor necrosis factor-alpha therapy, and combination thereof.

19. A method for treating heart failure with preserved ejection fraction using a combination therapy, the method comprising:
providing to a subject a therapeutically effective amount of a first compound that upregulates the Nrf2 pathway, inhibits NFκB, or increase adiponectin; and
administering a second agent that does not upregulate the Nrf2 pathway, inhibit NFκB, or increase adiponectin,
wherein the first compound is dimethyl fumarate, and
wherein the second agent is selected from the group consisting of: a diuretic, an ace-inhibitor, a beta-blocker, an angiotensin receptor blocker, isosorbide dinitrate, hydralazine, an aldosterone inhibitor, an angiotensin receptor neprilysin inhibitor, a PDE5 inhibitor, a statin, a neprilysin inhibitor, an antitumor necrosis factor-alpha therapy, and a combination thereof.

20. The method of claim 19, comprising administering dimethyl fumarate at a dose of 120 mg to 720 mg and a statin at a dose of 10 mg to 80 mg.
